(19) United States
(12) Patent Application Publication
(10) Pub. No.: US 2007/0275941 A1
(43) Pub. Date: Nov. 29, 2007

(54) SALT SENSITIVITY AND PREVENTION OF HYPERTENSION WITH DROSPIRENONE

(76) Inventor: Vladimir Hanes, Tarrytown, NY (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/749,592

(22) Filed: May 16, 2007

Related U.S. Application Data
(60) Provisional application No. 60/800,834, filed on May 17, 2006.

(51) Int. Cl. A61K 31/57; A61K 31/56

(52) U.S. Cl. 514/170

(57) ABSTRACT

The present invention relates to the use of drospirenone for manufacture of a medicament for prevention of development of high blood pressure (>140/90 mm Hg) in a patient who is predisposed to develop high blood pressure. The present invention further refers to methods of prevention of development of high blood pressure (≧140/90 mm Hg) in a patient who is predisposed to develop high blood pressure by administering drospirenone to a patient in need of such prevention.

This application claims the benefit of the filing date of U.S. Provisional Application Ser. No. 60/800,834 filed May 17, 2006, which is incorporated by reference herein.

The present invention refers to the use of drospirenone (DRSP) for manufacture of a medicament for prevention of development of high blood pressure (≧140/90 mm Hg, defined as hypertension) in a patient who is predisposed to develop high blood pressure (defined as prehypertension).

The present invention further refers to methods of prevention of development of high blood pressure (≧140/90 mm Hg) in a patient who is predisposed to develop high blood pressure by administering drospirenone to a patient in need of such prevention.

It is known that some subjects (termed as ‘salt sensitive’) will respond to a high sodium ingestion with a provable increase in blood pressure (BP), whereas others (termed as ‘salt resistant’) react with little or no BP changes (Myron H. Weinberger, Salt Sensitivity of Blood Pressure in Humans, Hypertension, 1996; 27: 481-490).

Salt sensitivity is found much more frequently in hypertensive (BP>=140/90 mmHg) and pre-hypertensive (BP=120-139/80-89 mmHg) subjects. Also, a presence of salt sensitivity predicts a considerably greater increase of BP with age, suggesting that salt sensitivity is important in the pathogenesis of hypertension. In addition, there is some epidemiological evidence demonstrating higher risk of cardiovascular disease (myocardial infarction, stroke, etc.) in subjects with salt sensitivity. Consequently, it would be important from clinical and public health perspectives to influence the phenomenon of prehypertension and salt sensitivity pharmacologically since this would prevent hypertension or delay progression of the prehypertension and salt sensitivity to hypertension.

It is thus a goal of the invention to provide a method of prevention of development of high blood pressure (>140/90 mm Hg) in a patient who is predisposed to develop high blood pressure and is in need of such prevention.

It was found that according to the invention this goal can be achieved by administering drospirenone to a patient in need of such prevention.

The pathogenesis of salt sensitivity is not fully understood; however, some investigations suggest that abnormally high aldosterone activity causing abnormal sodium and potassium handling by the kidney may be the cause of sodium sensitivity and essential hypertension.

Drospirenone is a novel progestin with anti-aldosterone activity which has been developed in combination with 17β-estradiol (E2) for use as a hormone therapy in postmenopausal women (WO 01/52857). In clinical studies drospirenone has demonstrated to consistently and substantially reduce blood pressure in postmenopausal women with hypertension either alone or in combination with other agents (WO 03/090755; Preston R A et al, AJH 2005; White W et al, Circulation 2005; White W et al, Hypertension 2006, in press). In addition, drospirenone has demonstrated a potassium sparing effect.

It was found that drospirenone surprisingly pharmacologically mitigates salt sensitivity of blood pressure due to its aldosterone receptor blocking and potassium-sparing effects, and thereby prevents or at least delays development of hypertension.

Drospirenone can be obtained from commercial sources (e.g., from Schering Aktiengesellschaft) or can by synthesized by conventional methods, e.g., according to the methods disclosed in U.S. Pat. No. 6,121,465 and Drugs of the Future 2000, 25 (12), 1247-1256.

Any of a variety of estrogens, as is well known in the art, and as they can be used for example in methods of hormone replacement therapy, can optionally be used together with drospirenone in the context of the present invention. Such estrogens include, e.g., ethinyl estradiol (EE), mestranol, estradiol (especially 17β-estradiol, known as E2) and esters thereof (e.g., valerate, acetate, benzoate or undecylate); estriol; estriol succinate; polyestriol phosphate; estrone; estrone sulfate; natural or synthetic estrogens; and conjugated estrogens.

DRSP and optionally an estrogen can be administered to a patient following conventional procedures, using conventional regimens of administration, kits, modes of administration, and dosages, all of which are well known to those of skill in the art.

Regimens are conventional and well known in the art for contraception and HRT purposes. The DRSP and estrogen can be administered concurrently, for any period of time, e.g., on a daily basis, 1-4 times a week, weekly, 2-3 weeks per month, etc. The two components can be administered separately (as disclosed, e.g., in U.S. Pat. No. 6,083,528), e.g., via a conventional kit, or as a combined preparation (e. g., a tablet or capsule).

The pharmaceutical compositions of the invention can be administered by any of a variety of conventional modes, including, e.g., oral (e. g, solutions, suspensions, tablets, dragees, capsules or pills), parenteral (including subcutaneous injection, or intravenous, intramuscular or intrasternal injection or infusion techniques), inhalation spray, transdermal, rectal, or vaginal (e.g., by vaginal rings or creams) administration. The two components can be administered by the same mode, or by different modes (e.g., transdermal estrogen and intravaginal DRSP).

According to the invention typical effective dosages for oral administration of drospirenone are about 0.25-3.0 mg/day. This range covers dosages typically used in drospirenone containing oral contraceptives (Yasmin®, Yaz®) and HRT products (Angeliq®).

A dosage that is “effective” to effect hormone replacement therapy is one that prevents or diminishes (alleviates) adverse physiological effects or symptoms resulting from reduced amounts of estrogen, such as, e.g., bone loss and resultant structural deformation, among many others. A dosage of a composition of the invention that is “effective” to reduce blood pressure is one that can achieve a measurable decrease in blood pressure. Any effective dosage can be administered in the methods of the invention, preferably a low dose formulation.

A dosage that is effective for contraception is typically 3.0 mg of drospirenone.

Effective dosages of estrogens are conventional and well known in the art. Typical approximate dosages for oral administration are, e.g., ethinyl estradiol (0.001-0.030 mg/day), mestranol (5-25 mcg/day), estradiol (including17β-estradiol), (0.5-6 mg/day), polyestriol phosphate (2-8 mg) and conjugated estrogens (0.3-1.2 mg/day). Dosages for other means of delivery will be evident to one of skill in the art. For example, transdermal dosages will vary therefrom in accordance with the absorption efficacy of the vehicle employed.

Equivalent dosages refer to doses which provoke comparable effects with respect to the effects on endometrium (contraceptive effects and cycle control for OCs), or comparable effects on vasomotor symptoms or prevention/treatment of osteoporosis (HRT).

Preferred combinations of the invention include, for oral administration, 3 mg DRSP/1 mg E2 and 2 mg DRSP/1 mg E2 or 3 mg DRSP/0.03 mg EE and 3 mg DRSP/0.02 mg EE.

It will be understood, of course, that the specific dose level and frequency of dosage for any particular patient will depend upon a variety of factors including the activity of the 15 specific compound employed, the metabolic stability and length of action of that compound, the age, body weight, general health, sex, diet, mode and time of administration, rate of excretion, drug combination, the severity of the particular condition, and the host undergoing therapy.

Because the method of this invention may involve effecting contraception or HRT, necessarily the individual doses of estrogen and DRSP are administered over a prolonged period of time, i. e., more than one month, usually at least several months and ordinarily for one or more years and often for one or more decades. During that period of time the size of the individual dose of either the estrogen or the DRSP or both can be changed at least once and often two or more times, usually stepwise increased in the case of the estrogen until the minimum effective therapeutic dosage is found. Often, it may be decreased again as the patient for example progresses from peri to post-menopause, because the estrogen dosage to prevent menopausal bone loss is usually higher than the dosage that is needed for effectively treating climacteric complaints.

Compositions of the invention can be formulated according to accepted pharmaceutical practice, with a conventional pharmaceutically acceptable vehicle, carrier, excipient, binder, preservative, stabilizer, flavor, and/or adjuvant, etc, for any given type of unit dosage form.

Formulations for oral administration are conventional in the art. For example, tablets generally contain a pharmaceutically acceptable carrier, e.g., a binder such as gum tragacanth, acacia, corn starch or gelatin; an excipient such as dicalcium phosphate or celluose; a disintegrating agent such as corn starch or alginic acid; a lubricant, such as magnesium stearate; and/or a sweetening agent or flavoring agent. When the dosage unit form is a capsule, it may contain in addition to materials of the above type a liquid carrier such as a fatty oil. Various other materials may be present as coatings or to otherwise modify the physical form of the dosage unit. For instance, tablets or capsules may be coated with shellac, sugar or both. A syrup or elixir may contain the active compound, water, alcohol or the like as the carrier, glycerol as solubilizer, sucrose as sweetening agent, methyl and propyl parabens as preservatives, a dye and a flavoring such as cherry or orange. When administered orally as a suspension, these compositions may contain microcrystalline cellulose for imparting bulk, alginic acid or sodium alginate as a suspending agent, methylcellulose as a viscosity enhancer, and sweetners/flavoring agents known in the art. As immediate release tablets, these compositions may contain microcrystalline cellulose, dicalcium phosphate, starch, magnesium stearate and lactose and/or other excipients, binders, disintegrants, diluents and lubricants known in the art.

Formulations suitable for injectable use include sterile aqueous solutions or dispersions and sterile powders for the extemporaneous preparation of sterile injectable solutions or dispersions. The solutions are stable and preserved against the contaminating action of microorganisms such as bacteria and fungi. The injectable solutions or suspensions may be formulated according to known art, using suitable non-toxic, parenterally-acceptable diluents or solvents, such as mannitol, 1,3 butanediol, water, Ringer's solution or isotonic sodium chloride solution, or suitable dispersing or wetting and suspending agents, such as sterile, bland, fixed oils, including synthetic mono-or diglycerides, and fatty acids, including oleic acid.

When rectally administered in the form of suppositories, these compositions may be prepared by mixing the drug with a suitable non irritating excipient, such as cocoa butter, synthetic glyceride esters or polyethylene glycols, which are solid at ordinary temperatures, but liquefy and/or dissolve in the rectal cavity to release the drug.

Methods of formulating HRT compositions for local application (e.g., as extrudable viscous liquids, semi-solid preparations such as gels, ointments or creams, or a spreadable solid such as a stick deodorant), and of applying them to a patient, e. g., to a surface such as skin or mucosa, are disclosed in U.S. Pat. No. 6,083,528.

In the foregoing and in the following examples, all temperatures are set forth uncorrected in degrees Celsius; and, unless otherwise indicated, all parts and percentages are by weight.

The effects of drospirenone/estradiol (Angeliq®) and medroxyprogesterone acetate/conjugated estrogen (Prempro™) on blood pressure and renal sodium handling in postmenopausal women with prehypertension are evaluated in a double blind, randomized, active-control study.

The primary aim of the study is to evaluate the effect of drospirenone/estradiol and medroxyprogesterone acetate/conjugated equine estrogens treatments on blood pressure in postmenopausal women with prehypertension over a period of 8 weeks. A secondary aim of the study is to investigate exploratory renal sodium handling in a subpopulation of the prehypertensive postmenopausal women.

Subjects:

90 (30 randomized subjects per treatment arm; 24 completers/treatment arm) menopausal women (≧1 year of menopause), age 45-60 years, with high-normal blood pressure (clinical SBP 130-139 mmHg or DBP 85-89 mmHg), and requiring HT are included in the study.

Exclusion Criteria Are:

Serum potassium>5.3 mEq/L (upper normal limit);
Serum creatinine>1.2 mg/dl or a creatinine clearance≦60 ml/min:
Known cardiovascular, renal or cerebral vascular disease, hypertension or heart failure;
Ingestion of diuretics, NSAIDs, steroids or other agents known to influence blood pressure, renal function or sodium handling.
   Blood Pressure Measurments:

Ambulatory blood pressure is monitored (ABPM) using a SpaceLabs monitoring device, office cuff blood pressure measurements using a calibrated sphygmomanometer, safety parameters including laboratory evaluations performed at a central laboratory, and 12-lead ECG evaluated by a central group.

Sodium Handling and Sodium Sensitivity Evaluations:

In a subgroup of approximately 18 subjects, sodium handling is evaluated using the following clinical research protocol: 3-day sodium loading period accomplished by a 175 ±25 mmol sodium dietary intake, followed by Na+excretion measured in 24-hour urine collection (Day 4), randomization to treatment (Day 5), followed by Na+excretion measured in 24 hour urine collection again on Day 6 and Day 7.

The Following Clinical Laboratory Tests Are Performed:

Hematology: red blood cell (RBC) and white blood cell (WBC) counts, hematocrit, hemoglobin, platelet and differential count

Serum Chemistry: glucose, blood urea nitrogen (BUN) creatinine, potassium, sodium, chloride, calcium, phosphorus, protein total, albumin, bilirubin total, alkaline phosphatase, aspartate aminotransferase (AST), alanine aminotransferase (ALT), cholesterol, triglycerides, high density lipoprotein (HDL), and low density lipoprotein (LDL)

Urinalysis: pH, specific gravity, glucose, white blood cell (WBC), red blood cell (RBC), and protein

Other tests: plasma renin activity, and serum aldosterone

At Screening (Visit 1), Week 4 (Visit 4), and Week 8B/Final B (Visit 6), hematology, serum chemistry, urinalysis, and plasma renin and serum aldosterone are performed. A cervical/vaginal smear will be performed at Screening (Visit 1), if necessary.

As Study Medication the Following Compositions Are Administered:

1. Angeliq: tablets containing 2 mg DRSP/1 mg E2, oral administration of 1 encapsulated tablet daily
2. Angeliq: tablets containing 0.5 mg DRSP/1 mg E2, oral administration of 1 encapsulated tablet daily
3. Prempro: tablets containing 1.5 mg MPA/0.3 mg CEE (conjugated equine estrogen), oral administration of 1 encapsulated tablet daily

All capsules look indistinguishable to keep the study double blind.

Primary Endpoint:

Change from Baseline to Week 8 in mean 24-hour systolic ABPM blood pressure
   Secondary Endpoints:
Change from Baseline to Week 8 in mean 24-hour diastolic ABPM blood pressure
Change from Baseline to Week 8 in systolic office cuff blood pressure at trough
Change from Baseline to Week 8 in diastolic office cuff blood pressure at trough
Change from Baseline to Week 8 in mean daytime (06:00 and 21:59) systolic ABPM blood pressure
Change from Baseline to Week 8 in mean daytime (06:00 and 21:59) diastolic ABPM blood pressure
Change from Baseline to Week 8 in mean nighttime (22:00 and 05:59) systolic ABPM blood pressure
Change from Baseline to Week 8 in mean nighttime (22:00 and 05:59) diastolic ABPM blood pressure
Mean change from Baseline to Week 8 in systolic ABPM blood pressure measured at trough
Mean change from Baseline to Week 8 in diastolic ABPM blood pressure measured at trough
Change from Baseline to Week 8 in mean body weight

Descriptive statistics of 24-hour sodium excretion are tabulated.

Specific Requirements:

ABPM is performed in all subjects to monitor the effect of the treatments during a 24-hour interval between the baseline visits and between the final visits. ABPM is performed using a Spacelabs 90207 device.

Office cuff blood pressure measurements are measured at trough (i.e., 24±3 hours after the previous dose). All office cuff blood pressure measurements are performed using a calibrated sphygmomanometer with an appropriate cuff size (cuff bladder encircling at least 80% of the arm) to ensure accuracy. All measurements are performed on the nondominant arm and while the subject is sitting. The first measurement takes place after at least 5 minutes of rest. The time of the first office cuff blood pressure measurement is recorded on the CRF as well as the 2 remaining individual measurements. The means of the 3 measurements separated by at least 2 minutes are calculated at each visit (mean of the 3 systolic readings/mean of the 3 diastolic readings).

Laboratory evaluations are done by a Central laboratory with established standard measurements of laboratory parameters.

Mean decreases from baseline in 24-hour systolic and diastolic Ambulatory Blood Pressure Monitoring (ABBP) and clinical blood pressure values during the DRSP/E2 treatment period in prehypertensive women are observed. At Week 8, prehypertensive women treated with 2 mg DRSP/1 mg E2 experience a significant decrease in systolic/diastolic blood pressure values. The blood pressure lowering effect is more pronounced with the higher DRSP dose. The effect is apparent within 2 weeks of DRSP/E2 treatment with maximum effect achieved within 6 weeks from the start of the therapy. In the Prempro treatment group, a slight increase of systolic/diatoc BP values is recoded.

The inventors therefore conclude that DRSP can prevent or delay development of high blood prerssure in a subject who is predisposed to develop high blood pressure (hypertension).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever. In the foregoing and in the examples, all temperatures are set forth uncorrected in degrees Celsius and, all parts and percentages are by weight, unless otherwise indicated.

The entire disclosures of all applications, patents and publications, cited herein and of corresponding U.S. Provisional Application Ser. No. 60/800,834, filed May 17, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1-13. (canceled)

14. A method of prevention of development of high blood pressure (≧140/90 mm Hg) in a patient who is predisposed to develop high blood pressure by administering drospirenone to a patient in need of such prevention.

15. The method according to claim 14 wherein the patient is a salt-sensitive patient.

16. The method according to claim 14 wherein the patient is a human.

17. The method according to claim 14 wherein the patient is a male human.

18. The method according to claim 16 wherein the patient is a female human.

19. The method according to claim 18 wherein an estrogen is administered in addition to the drospirenone.

20. The method according to claim 19 wherein the estrogen is ethinyl estradiol, mestranol, quinestranol, estradiol, estrone, estrane, estriol, estetrol or conjugated equine estrogens.

21. The method according to claim 20 wherein the estrogen is estradiol.

22. The method according to claim 20 wherein the estrogen is a conjugated equine estrogen (CEE).

23. The method according to claim 20 wherein the estrogen is ethinyl estradiol.

24. The method according to claim 14 wherein the amount of drospirenone administered is from 0.25 mg to 3.0 calculated on a daily basis.

25. The method according to claim 24 wherein the amount of drospirenone is from 0.25 mg to 3.0 mg calculated per daily dosage unit.

26. The method according to claim 19 wherein the amount of the estrogen is up to 2.0 mg of estradiol or an bioequivalent amount of another estrogen.
